(19) United States
(12) Patent Application Publication
(10) Pub. No.: US 2024/0423460 A1
(43) Pub. Date: Dec. 26, 2024

(54) MEDICAL SYSTEMS, DEVICES, AND RELATED METHODS

(71) Applicant: Boston Scientific Scimed, Inc., Maple Grove, MN (US)

(72) Inventors: Jimmy HARRISON, Greencastle, IN (US); Thomas M. RAGONESE, Bloomington, IN (US); Camron HAGEMEYER, Bloomington, IN (US); Mark Allan VOSS, Spencer, IN (US); Daniel Joseph GRASSO, Franklin, MA (US)

(73) Assignee: Boston Scientific Scimed, Inc., Maple Grove, MN (US)

(21) Appl. No.: 18/753,508

(22) Filed: Jun. 25, 2024

Related U.S. Application Data
(60) Provisional application No. 63/510,201, filed on Jun. 26, 2023.

(51) Int. Cl. A61B 1/018; A61B 1/00; A61B 1/005; A61B 1/06

(52) U.S. Cl. CPC A61B 1/018; A61B 1/00094; A61B 1/00096; A61B 1/00101; A61B 1/00105; A61B 1/005; A61B 1/0676

(57) ABSTRACT

Medical devices and related methods are described, including a medical device that includes a shaft defining at least one working channel extending from a proximal end of the shaft to a distal end of the shaft along a longitudinal axis and a distal tip coupled to the shaft. The working channel may have a cross-sectional dimension that allows passage of an instrument therethrough. The distal tip may include a first arm and a second arm, the first arm being moveable relative to the second arm between a first configuration that obstructs a portion of the working channel and a second configuration that provides unobstructed access to the working channel, e.g., to allow the instrument to extend distal to the medical device.

### CROSS-REFERENCE TO RELATED APPLICATION(S)

This patent application claims the benefit of priority to U.S. Provisional Application No. 63/510,201, filed on Jun. 26, 2023, which is incorporated by reference herein in its entirety.

### TECHNICAL FIELD

Various aspects of this disclosure relate generally to medical systems, devices, and methods useful in medical procedures. In particular, medical systems, devices, and methods for accessing a body lumen and/or delivering medical instruments to a body lumen.

### BACKGROUND

During medical procedures, such as endoscopy and/or ureteroscopy procedures, an operator may insert a medical device into a patient, and guide that medical device through tortuous anatomy for positioning the device at a target treatment site in the body. Accessing anatomy of varying sizes may present challenges to the device accessing the target treatment site.

### SUMMARY

The present disclosure includes medical systems, devices, and methods useful in medical procedures, e.g., endoscopic procedures. According to some aspects of the present disclosure, the medical device may include a shaft defining at least one working channel extending from a proximal end of the shaft to a distal end of the shaft along a longitudinal axis, the working channel may have a cross-sectional dimension that allows passage of an instrument therethrough, and a distal tip coupled to the shaft and may include a first arm and a second arm, the first arm may be moveable relative to the second arm between a first configuration that may obstruct a portion of the working channel and a second configuration that may provide unobstructed access to the working channel to allow the instrument to extend distal to the medical device.

According to some aspects, a cross-sectional dimension of the working channel at the distal end of the shaft may be at least 50% of a cross-sectional dimension of the distal end of the shaft. In some examples, the first arm may be moveable relative to the second arm via a hinge. In some examples, the hinge may include a leaf spring or a torsion spring. In some examples, the hinge may be a living hinge, the first arm being integral with the second arm and moveable via the living hinge. In some examples, the distal tip may be biased to the first configuration. In some examples, the distal tip may include at least one of an imaging device or a light source and the shaft may further include at least one lumen containing an electronic connection to the at least one of the imaging device or light source. In some examples, the at least one of the imaging device or light source may be on a distal-most surface of the distal tip. In some examples, one of the first arm or the second arm may include an imaging device and the other of the first arm or the second arm may include a light source. In some examples, the shaft may further include a lumen for irrigation or suction, and the lumen may extend through the distal tip to a distal opening in the first arm or the second arm. In some examples, the cross-sectional dimension of the working channel may be at least 0.1 cm. In some examples, a cross-sectional dimension of the shaft may be greater than 0.1 cm and less than 0.3 cm. In some examples, the working channel may be defined by a tube moveable relative to the shaft and the distal tip. In some examples the medical device may further include a flexible or elastic sleeve around at least a portion of the distal tip. In some examples, the distal tip may be detachable from the shaft via complementary mating features of the distal tip and the shaft.

According to some aspects of the present disclosure, a medical device may include a shaft defining at least one working channel extending along a longitudinal axis from a proximal end of the shaft to a distal end of the shaft, and a distal tip coupled to the shaft and including a first arm and a second arm, the first arm may be moveable relative to the second arm via a hinge between a first configuration that may obstruct a portion of the working channel and a second configuration that may provide unobstructed access to the working channel to allow an instrument to extend distal to the medical device, the distal tip may be biased to the first configuration.

According to some aspects, the working channel may extend through the distal tip between the first arm and the second arm. In some examples, the distal tip may include an imaging device and a light source each disposed on a distal-most surface of the distal tip.

According to some aspects of the present disclosure, a medical device may include a shaft defining at least one working channel extending from a proximal end of the shaft to a distal end of the shaft, and a distal tip coupled to the shaft and including a first arm and a second arm, wherein each of the first arm and the second may include one of a light source or an imaging device at a distal end of the respective first or second arm, and wherein the first arm may be moveable relative to the second arm between a first configuration that may obstruct a portion of the working channel and a second configuration that may provide unobstructed access to the working channel. In some examples, the medical device may further include a flexible or elastic sleeve around at least a portion of the distal tip.

### BRIEF DESCRIPTION OF FIGURES

The accompanying drawings, which are incorporated in and constitute a part of this application, illustrate exemplary aspects of this disclosure and together with the description, serve to explain the principles of the present disclosure.

FIG. 1 illustrates an exemplary medical device, in accordance with some aspects of this disclosure.

FIGS. 2A-2D illustrate features of the distal end portion of an exemplary medical device in a first configuration, in accordance with some aspects of the present disclosure.

FIGS. 3A-3D illustrate features of the distal end portion of the medical device of FIGS. 2A-2D in a second configuration, in accordance with some aspects of the present disclosure.

FIG. 4 illustrates features of an exemplary medical device, in accordance with some aspects of this disclosure.

FIGS. 5A and 5B illustrate an exemplary medical device, in accordance with some aspects of this disclosure.

FIGS. 6A and 6B illustrate an exemplary medical device, in accordance with some aspects of the present disclosure.

FIG. 7 illustrates an exemplary medical device, in accordance with some aspects of this disclosure.

FIGS. 8A-8C illustrate an exemplary medical device, in accordance with some aspects of the present disclosure.

FIGS. 9A and 9B illustrate an exemplary medical device, in accordance with some aspects of this disclosure.

FIG. 10 illustrates an exemplary medical device, in accordance with some aspects of the present disclosure.

### DETAILED DESCRIPTION

Particular aspects of the present disclosure are described in greater detail below. The terms and definitions provided herein control, if in conflict with terms and/or definitions incorporated by reference.

The terms “proximal” and “distal” are used herein to refer to the relative positions of the components of exemplary medical devices. As used herein, “proximal” refers to a position relatively closer to the exterior of the body or closer to an operator using the medical device. In contrast, “distal” refers to a position relatively further away from the operator using the medical device, or closer to the interior of the body.

As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.”

Further, relative terms such as, for example, “about,” “substantially,” “approximately,” etc., are used to indicate a possible variation of ±10% in a stated numeric value or range.

Aspects of the present disclosure include medical systems, devices, and methods for improving accessibility to anatomy of varying sizes during a medical procedure, among other aspects.

Although ureteroscopes and endoscopes are referenced herein for illustration purposes, it will be appreciated that the disclosure encompasses any suitable medical device configured to allow an operator to access and view internal body anatomies of a patient and/or to deliver medical instruments, such as, for example, biopsy forceps, graspers, baskets, snares, probes, scissors, retrieval devices, lasers, other tools, into the patient's body. The medical device may be inserted into a variety of body lumens and/or cavities, such as, for example, any portion of a urinary tract including a ureter, a gastrointestinal lumen including an esophagus, a vascular lumen, an airway, and the like. It will be appreciated that, unless otherwise specified, bronchoscopes, duodenoscopes, endoscopes, gastroscopes, endoscopic ultrasonography (“EUS”) scopes, colonoscopes, ureteroscopes, bronchoscopes, laparoscopes, cystoscopes, aspiration scopes, sheaths, catheters, or any other suitable delivery device or medical device may be used in connection with the features described herein.

The medical devices and systems herein may provide for a reduced profile to assist with navigating through tortuous body anatomy, while still providing sufficient room for various components at or proximate the distal end (e.g., imaging devices, light sources, sensors, channels, etc.). For various medical procedures, it may be desirable to have certain features on or proximate the distal end face of the device, e.g., to illuminate and image different features (e.g., light source(s), camera(s), etc.), to collect data using sensors, and/or to access one or more channels (e.g., working channel(s), irrigation/aspiration/fluid channel(s), suction channel(s), etc.). Furthermore, having one or more of the components (e.g., working channels(s)) proximal to the distal end face of the device and one or more components (e.g., light sources(s), camera(s)) on the distal end face of the device may allow for each of the components to have a cross-sectional dimension larger than a cross-sectional dimension of components on a distal end face of other medical devices. According to some aspects of the present disclosure, the medical device may have a reduced outer diameter as compared to other medical devices, the distal end portion including a mechanism for selectively accessing one or more working channels by expanding the outer profile of the distal end portion.

The working channel(s) of the shaft may have a cross-sectional dimension large enough to allow for passage of an instrument therethrough, while maintaining a relatively low profile of the shaft and distal tip to facilitate navigation through patient anatomy. For example, the cross-sectional dimension (e.g., diameter) of the working channel may be at least 0.1 cm. In some examples, the cross-sectional dimension of the lumen at the distal end of the shaft may be at least 30%, at least 50%, at least 60%, at least 70%, or at least 80% of the cross-sectional dimension of the distal end of the shaft. Further, for example, the cross-sectional dimension of the shaft may be greater than 0.1 cm and less than 0.3 cm.

For example, the distal end portion of the medical device may transition between a first configuration (e.g., closed configuration) suitable for navigation through a bodily lumen and a second configuration (e.g., open configuration) suitable for delivery of an instrument through a working channel of the medical device at a target treatment site, wherein the distal end portion of the medical device has a cross-sectional dimension in the first configuration less than the cross-sectional dimension of the distal end portion in the second configuration. While in both the closed configuration and the open configuration, the distal end portion, e.g., distal tip, may include at least one electronic component facing in a distalmost direction of the medical device, e.g., located on or integrated into a distalmost surface of the distal tip (e.g., a distalmost surface of one or both arms of the distal tip). Such electronic components may include, for example, an imaging device, a light source, and/or a sensor. Lumens within the shaft and/or the distal tip may contain an electronic connection to electronic component(s), e.g., housing wires and/or cables to supply power and allow for control of the component(s). The electronic one of the first arm or the second arm includes an imaging device and the other of the first arm or the second arm includes a light source.

The medical devices and systems herein may facilitate access to internal patient anatomy and may present less trauma and/or risk of trauma to patient tissues, e.g., in view of the reduced outer diameter, while maintaining the ability to utilize various electronic components at or proximate the distal end.

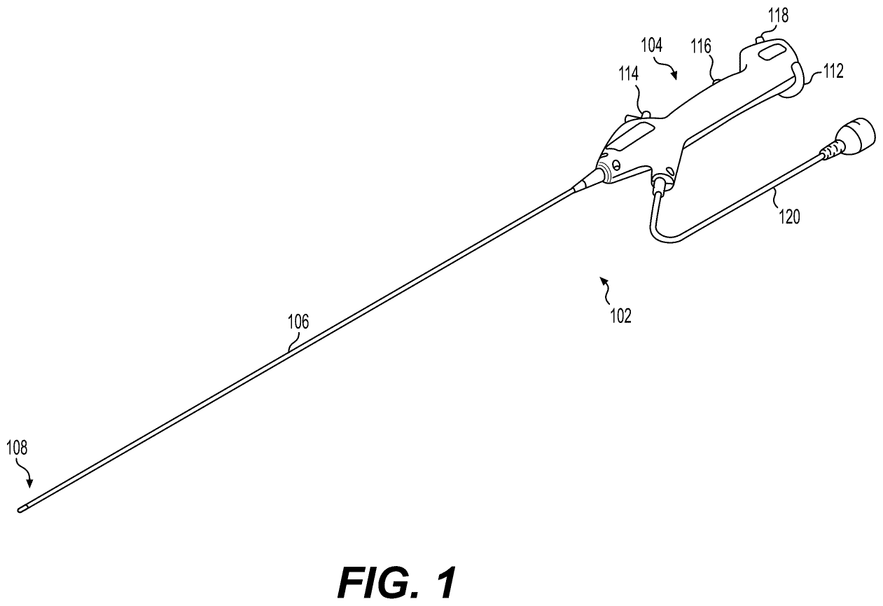

An exemplary medical device 102 (e.g., ureteroscope) is shown in FIG. 1. Medical device 102 as shown includes a handle 104 and a shaft 106 extending distally from handle 104. Shaft 106 may be flexible to facilitate navigation of shaft 106 through tortuous anatomical passages in a patient's body. Shaft 106 may include or be coupled to a distal end portion that terminates at a distal tip 108. Medical device 102 may include an actuator 112, for example, on handle 104, to facilitate articulation, steering, deflection, and/or other movement of shaft 106, including distal tip 108.

Medical device 102 may also include one or more lumens extending through shaft 106, and one or more openings, e.g., proximate and/or distal openings, in communication with the one or more lumens. For example, the one or more lumens may extend through handle 104 and/or shaft 106, and the one or more openings may be in handle 104 and/or distal tip 108.

For example, handle 104 may include a port 114 in communication with a lumen of medical device 102. In some aspects, port 114 may be a T-shape port, and/or may include one or more valves, Luer connections, etc. An operator may insert an instrument or other device into port 114 and may extend the instrument or other device distally through the lumen of medical device 102. Handle 104 may also include a valve 116, e.g., suction valve. FIG. 1 shows valve 116 on an opposing side from actuator 112. Valve 116 may be in communication with the same lumen as port 114 or a different lumen, and may be used to control suction, e.g., to draw material through the lumen. Handle 104 may also include a second actuator 118, e.g., a button for image capture. Actuator 118 may allow an operator to capture video and/or still images from an imaging device at distal tip 108. Medical device 102 may also include a light source and/or camera at distal tip 108. In some examples, distal tip 108 is integral with the shaft 106. In other examples, shaft 106 and distal tip 108 are separate pieces coupled together. For example, distal tip 108 may comprise an end cap fixedly or detachably coupled to the distal end of shaft 106.

Handle 104 may be coupled to an umbilicus 120. Umbilicus 120 may extend from handle 104 to one or more auxiliary devices. The one or more auxiliary devices may include a controller or control system, an imaging system, a power supply, a fluid supply, a suction/vacuum source, a display, etc.

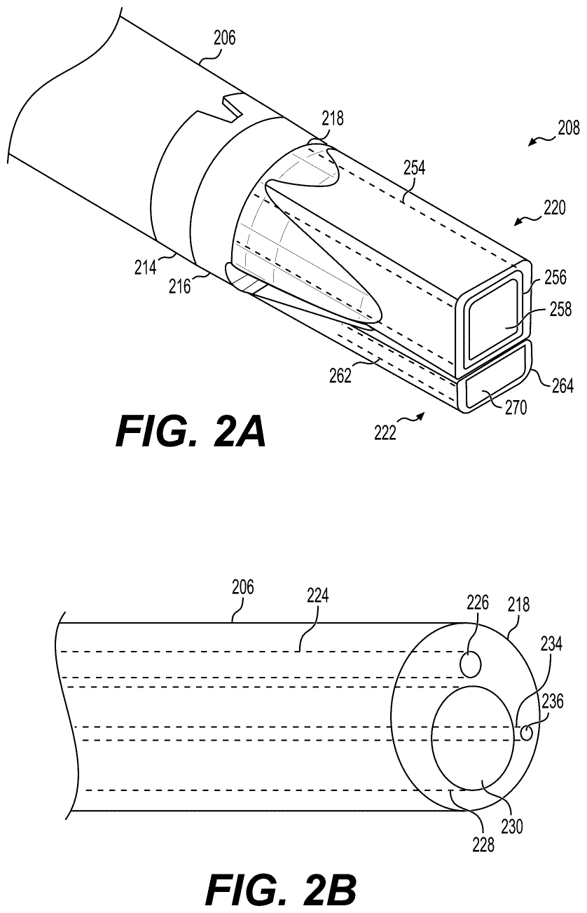

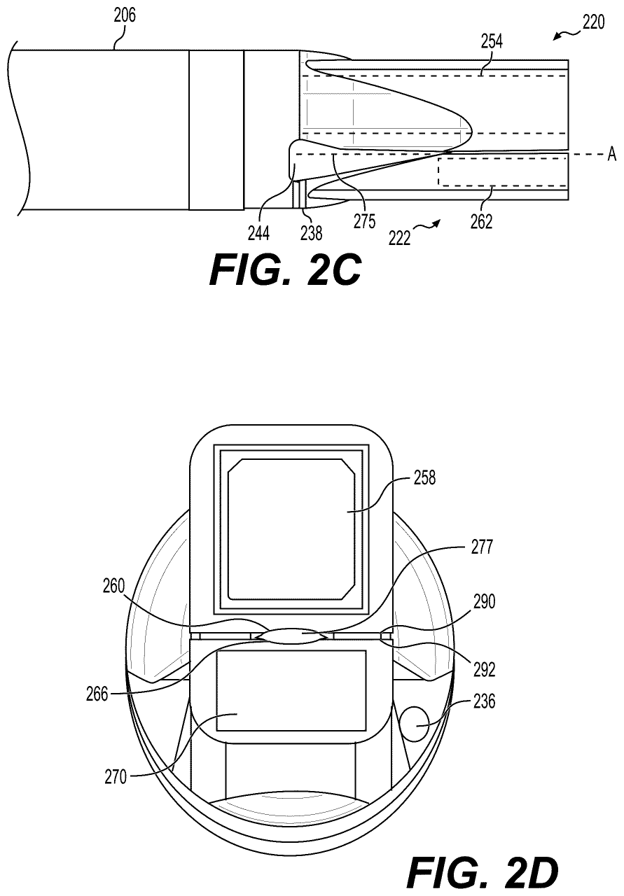

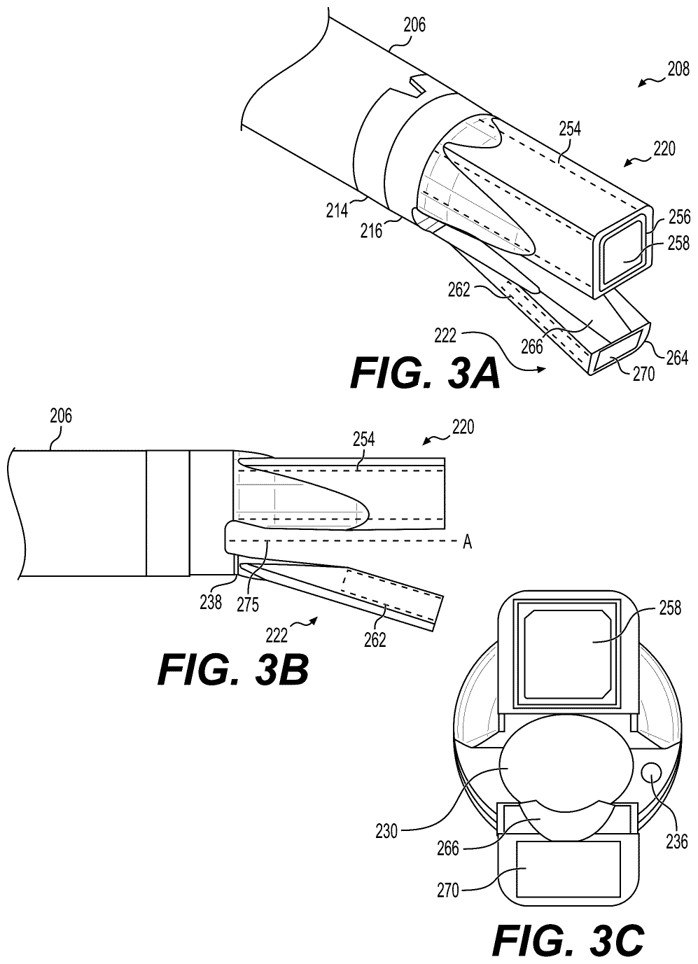

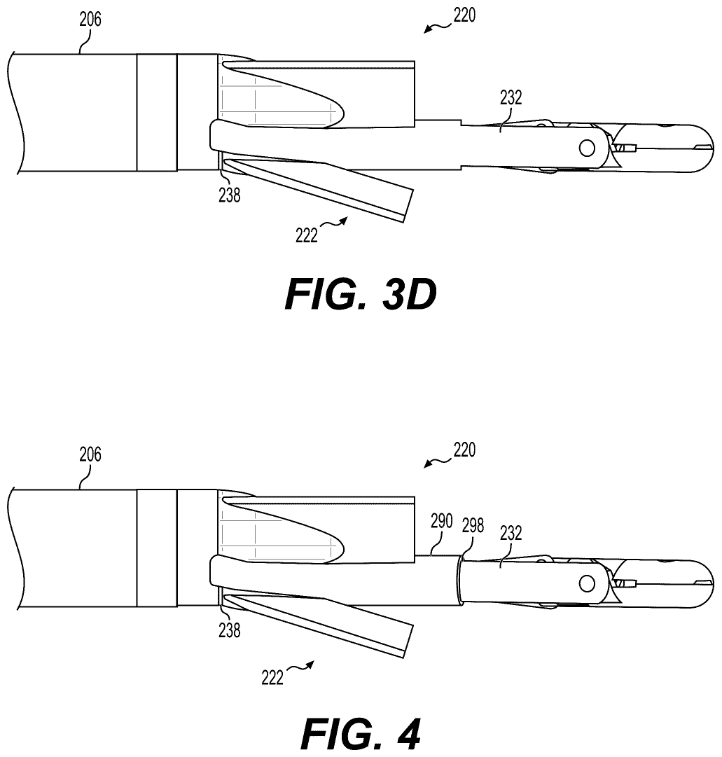

FIGS. 2A-2D and 3A-3D illustrate the distal end portion of an exemplary medical device according to aspects of the present disclosure, the distal end portion including shaft 206 and distal tip 208. FIGS. 2A, 2C, and 2D show a first, closed configuration of distal tip 208, and FIGS. 3A-3D show a second, open configuration of distal tip 208. The distal end portion (including distal tip 208) of the medical device has a cross-sectional dimension in the first, closed configuration less than the cross-sectional dimension of the distal end portion in the second, open configuration The medical device illustrated in FIGS. 2A-2D may include any of the features of medical device 102 of FIG. 1, including any features of shaft 106 and/or distal tip 108. In this example, shaft 206 and distal tip 208 are separate pieces coupled or attached together, e.g., fixedly or detachably coupled together. For example, the distal end of shaft 206 is shown coupled to the proximal end of distal tip 208. Optionally, distal tip 208 includes a first portion 214 and/or a second portion 216 configured to attach to the distal end of shaft 206. For example, first portion 214 includes a notch that may be complementary to a groove or tab of the distal end of shaft 206. In the event the shaft has a circular cross-section, first portion 214 and second portion 216 may be cylindrical in shape. First portion 214 may have an outer cross-sectional dimension (e.g., outer diameter) approximately equal to an outer cross-sectional dimension (e.g., outer diameter) of shaft 206, and second portion 216 may have an outer cross-sectional dimension (e.g., outer diameter) equal to or less than an outer cross-sectional dimension (e.g., outer diameter) of first portion 214. In some examples, second portion 216 may have an outer cross-sectional dimension (e.g., diameter) equal to the outer cross-sectional dimension (e.g., outer diameter) of shaft 206 and first portion 214. In some examples, first portion 214 and second portion 216 may be integral with one another and the remainder of distal tip 208 to form a single body (e.g., a single integral end cap).

According to some aspects of the present disclosure, shaft 206 may define at last one working channel extending from a proximal end of shaft 206 to a distal end of shaft along a longitudinal axis, the working channel having a cross-sectional dimension that allows passage of an instrument therethrough. FIG. 2B shows the distal end of shaft 206 to illustrate lumens or channels that may extend through shaft 206. Shaft 206 may include a distal face 218. Distal face 218 may be circular in cross-section (as shown) or any other shape, according to the cross-sectional shape of shaft 206. Distal face 218 may define one or more openings in communication with respective lumens or channels in shaft 206. For example, distal face 218 as shown defines first opening 226, second opening 230, and third opening 236 in communication with respective lumens, e.g., first lumen 224, second lumen 228, and third lumen 234. The lumens 224, 228, 234 may allow for passage of an instrument, fluid and/or suction, or wires or cabling to control and power electronic components at the distal tip 208. For example, second lumen 228 may be relatively larger than the other lumens in order to serve as a working channel (working channel 228) and facilitate passage of an instrument therethrough. First lumen 224 and third lumen 234 may be relatively smaller than working channel 228, e.g., each of first lumen 224 and third lumen 234 being suitable for use as an aspiration/suction channel or to connect electronic component(s) of the distal tip 208 to a proximal power source and/or controller via wires of cables.

As mentioned above, the present disclosure includes mechanisms for selectively accessing one or more working channels of a medical device by expanding the outer profile of the distal end portion of the medical device. Referring to FIGS. 2A, 2C, and 2D, distal tip 208 may include a first arm 220 and a second arm 222 coupled together and moveable relative to each other, e.g., towards and away from each other. In some examples, the arms 220, 222 are integral with one other via a living hinge to allow movement, or the arms 220, 222 are separate pieces coupled together via a hinge. First arm 220 and second arm 222 may comprise one or more biocompatible materials and may be flexible, semi-flexible, or non-flexible materials.

Space between the first arm 220 and second arm 222 when the arms 220, 222 move apart from each other may provide access to working channel 228. In some examples, at least a portion of the distal tip 208 may define a portion of working channel 228. For example, an inner surface of first arm 220 and/or an inner surface of second arm 222 may be concave and form respective channels 260, 266, e.g., as a portion of or continuation of working channel 228 along axis A parallel to the longitudinal axis of shaft 206. In some examples, channels 260, 266 may taper in a direction from a proximal end to a distal end of distal tip 208. Leveled portions 290, 292 of respective inner surfaces of first arm 220 and second arm 222 may extend alongside edges of channels 260, 266. Proximal ends of channels 260, 266 may be in alignment with opening 230. Edges of channels 260, 266 may extend longitudinally parallel to one another, such that channels 260, 266 define a semi-enclosed space or passageway 275 when inner surfaces of first arm 220 and second arm 222 are in close proximity to one another in a closed configuration of distal tip 208. In some examples, leveled portions 290, 292 at distal ends of arms 220, 222 may contact one another in the closed configuration. In some examples, channels 260, 266 may form a distal-facing opening 277 in the closed configuration. Passageway 275 may be generally funnel-shaped when distal tip 208 is in the closed configuration. For example, an outer cross-sectional dimension at a proximal end of passageway 275 may be larger than an outer cross-sectional dimension at a distal end of passageway 275. For example, distal-facing opening 277 may have a smaller cross-sectional dimension than a cross-sectional dimension of opening 230 of distal face 218.

Distal tip 208 may include a mechanism to transition between a first configuration that at least partially obstructs a portion of working channel 228 of shaft 206 and a second configuration that provides unobstructed access to working channel 228, e.g., to allow an instrument to pass through distal tip 208 and extend distal to the medical device. For example, the instrument may include a forceps, grasper, basket, snare, probe, scissor, retrieval devices laser, etc., used to treat a target site of patient tissue. The mechanism may include moving the first arm 220 relative to the second arm 222 between the closed configuration and open configuration. For example, first arm 220 may move relative to second arm 222 while second arm 222 remains stationary relative to shaft 206, second arm 222 may move relative to first arm 220 while first arm 220 remains stationary relative to shaft 206, or both first arm 220 and second arm 222 may move relative to each other and relative to shaft 206.

First arm 220, second arm 222 and/or both may define one or more lumens. The lumen(s) may be closed at the distal end of distal tip 208 or may be open (e.g., allowing instruments to exit medical device and/or allowing for aspiration or suction at a target site of patient tissue). Referring to FIGS. 2A, 2C, and 2D, first arm 220 may include at least one lumen, e.g., lumen 254 in communication with a corresponding lumen of shaft 206, e.g., first lumen 224. For example, when shaft 206 and distal tip 208 are coupled together, first lumen 224 of shaft 206 may be in communication with lumen 254 of distal tip 208 to form a continuous channel. Lumen 254 (and first lumen 224) may be used to provide electronic connection (e.g., carrying wire(s), cable(s), etc.) to one or more imaging devices 258 (e.g., camera(s), imager(s), etc.) of distal tip 208. For example, distal tip 208 may include one or more imaging devices 258 coupled to a power source, controller, display, etc., via one or more wires, etc., within lumens 224, 254 to a handle and/or umbilicus of the medical device (e.g., similar to handle 104 and/or umbilicus 120 of medical device 102).

Additionally or alternatively, second arm 222 may include at least one lumen, e.g., lumen 262 in communication with a corresponding lumen of shaft 206, e.g., third lumen 234. For example, when shaft 206 and distal tip 208 are coupled together, third lumen 234 of shaft 206 and lumen 262 of distal tip 208 may be in communication to form a continuous channel. Lumen 262 (and third lumen 234) may be used to provide electronic connection (e.g., carrying wire(s), cable(s), etc.) to one or more light sources 270 (e.g., LEDs, fiber optic cables, light guides, etc.) of distal tip 208. For example, distal tip 208 may include at least one light source configured to illuminate the treatment site. The light source(s) 270 may be coupled to a power source, controller, display, etc., via one or more wires (e.g., optic cables, etc.) within lumens 234, 262 to a handle and/or umbilicus of the medical device (e.g., similar to handle 104 and/or umbilicus 120 of medical device 102).

Second arm 222 may be moveable relative to first arm 220 via a hinge 238 between a first, closed configuration (e.g., FIG. 2A) and a second, open configuration (e.g., FIG. 3A). For example, first and second arms 220, 222 may move relative to each other about a pivot axis. In some examples, second arm 222 may be moveable independent of first arm 220. Hinge 238 may allow second arm 222 to move away from central axis A and away from first arm 220 to the open configuration of distal tip 208, and/or to move towards central axis A (see FIGS. 3A-3D) and towards first arm 220 to the closed configuration (see FIGS. 2A-2D). While in the first, closed configuration, distal tip 208 may at least partially obstruct working channel 228 of shaft 206, while in the second, open configuration, distal tip 208 may expose an entirety or a substantial entirety of working channel 228. Hinge 238 may be a living hinge (e.g., if first arm 220 and second arm 222 are integral with each other to form a single piece) or may include a spring such as a torsion spring or leaf spring. Hinge 238 may bias second arm 222 towards axis A (closed configuration). Hinge 238 may allow for second arm 222 to remain in the closed configuration as distal tip 208 passes through tight portions of patient anatomy.

As previously mentioned, instruments may be passed through working channel 228 of shaft 206. As shown in FIG. 3D, an instrument 232 may be extended through working channel 228 of shaft 206 towards distal tip 208. While distal tip 208 is in the closed configuration (e.g., FIG. 2C), there may be insufficient space between first and second arms 220, 222 to permit instrument 232 to exit distal tip 208. Although working channel 228 is obstructed, this closed configuration may maintain a relatively low profile of the medial device to facilitate navigation through patient anatomy, e.g., before instrument 232 will be used for treatment. Upon reaching a target site, the distal tip 208 may transition to the open configuration of FIG. 3D to expose working channel 228 and allow instrument 232 to exit distal tip 208 through passageway 275 between first and second arms 220, 222. In some examples, instrument 232 may be advanced distally to contact inner surfaces of first arm 220 and second arm to move second arm 222 away from axis A into the open configuration, e.g., while first arm 220 remains fixed relative to shaft 206. For example, as instrument 232 is moved distally within passageway 275, instrument 232 applies a force on second arm 222 to move second arm 222 away from axis A and away from first arm 220. Instrument 232 may be extended distally beyond the distal end of distal tip 208, e.g., for treatment at a target site of patient tissue. Second arm 222 may be biased towards the first, closed configuration and move towards first arm 220 and towards axis A to the closed configuration as instrument 232 is retracted proximally through passageway 275.

Optionally, shaft 206 may include a tube capable of moving longitudinally to facilitate advancing and retracting instrument 232 within working channel 228 of shaft 206 and within distal tip 208. For example, as shown in FIG. 4, shaft 206 is shown with a retractable tube 290 defining a lumen 298 housed within working lumen 228. An operator of the medical device may control translation of tube 290 along shaft 206 by a suitable mechanism and actuator, e.g., a lever, switch, button, or other actuator on a handle of the medical device (e.g., handle 104 shown in FIG. 1). For example, the medical device including shaft 206 and distal tip 208 may be inserted into a natural body orifice and advanced to a target site. The operator may engage the actuator to move tube 290 distally through passageway 275 of distal tip 208. Similar to the discussion above regarding distal movement of instrument 232, distal movement of tube 290 through passageway 275 may move second arm 222 away from axis A to the open configuration of distal tip 208. In some examples, tube 290 may be moved distally beyond the distal end of distal tip 208.

An instrument 232 may be translated through lumen 298 of tube 290 and distally beyond a distal end of tube 290. Tube 290 may allow for instrument 232 to be delivered to a target site unimpeded by first and second arms 220, 222. The operator may retract instrument 232 proximally through lumen 298 of tube 290 while maintaining distal tip 208 in the open configuration. The operator may then engage the actuator to move tube 290 proximally such that a distal end of the tube 290 is retracted within shaft 206. Proximal movement of tube 290 through passageway 275 may remove the force maintaining second arm 222 away from first arm 220, such that second arm 222 moves towards axis A to the closed configuration of distal tip 208.

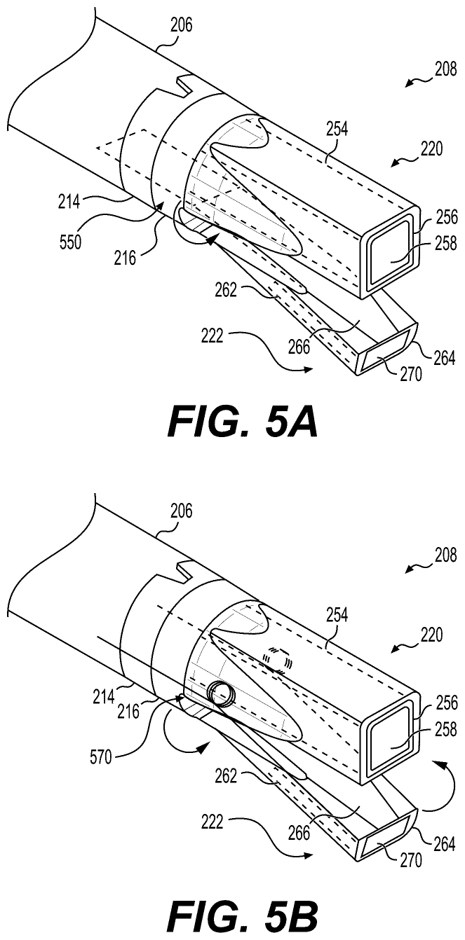

FIGS. 5A and 5B illustrate exemplary hinges that may be used in the medical device shown in FIGS. 2A-2D, 3A-3D, and 4. As shown, second arm 222 is coupled to shaft 206 via hinges including spring mechanisms for moving second arm 220 away from axis A to the open configuration (FIG. 3A) and towards axis A to the closed configuration (FIG. 2A). Second arm 220 may be coupled to shaft 206 via a leaf spring 550, as shown in FIG. 5A, or a torsion spring 570, as shown in FIG. 5B. For example, second arm 222 may pivot relative to first arm 220 about a pivot axis defined by the hinges.

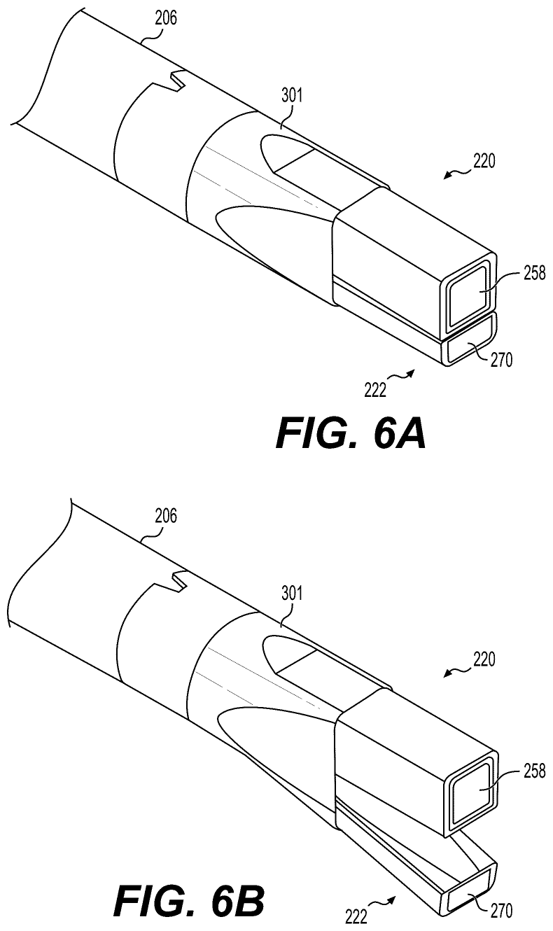

In some examples, at least a portion of distal tip 208 may include a flexible outer covering, e.g., to protect distal tip 208 and/or surrounding patient anatomy during use. As shown in FIGS. 6A and 6B, a band or sleeve 301 may surround a proximal portion of first and second arms 220, 222 of distal tip 208. Sleeve 301 may extend from proximal end of distal tip 208 including proximal ends of first and second arms 220, 222 to partially cover or completely cover first and second arms 220, 222, depending on the length of sleeve 301. In other words, sleeve 301 may cover a portion or an entirety of the periphery of distal tip 208. Sleeve 301 may enclose side openings formed between arms 220, 222, and/or any side openings formed when second arm 222 is in the open configuration. For example, sleeve 301 may enclose gap 244 (see FIG. 2C) formed between arms 220, 222 in the open and closed configurations. Sleeve 301 may be elastic and flexible to permit transition between the closed and open configurations of distal tip 208 and/or to bias first and second arms 220, 222 to the closed configuration absent force provided therebetween to move the arms 220, 222 apart.

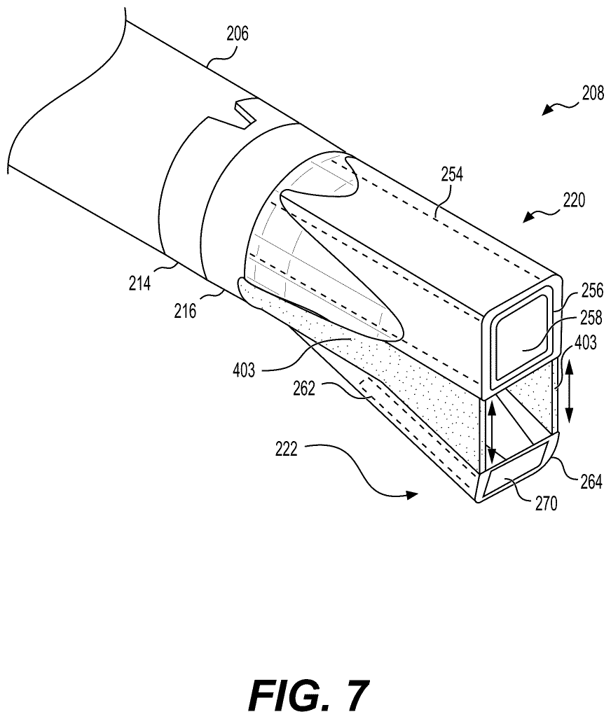

In another variation, the distal tip 208 may include a material between first and second arms 220, 222, in addition to or as an alternative to sleeve 301. For example, FIG. 7 shows overmolds 403, 403′ between side inner surfaces of first and second arms 220, 222, opposite one another. Overmolds 403, 403′ may enclose side openings formed between first and second arms 220, 222 in the open and closed configurations. Such features may inhibit inadvertent passage of material through the side openings (e.g., fluids and/or tissue from patient anatomy into the medical device and/or instruments or fluid from the medical device into surrounding patient anatomy).

Sleeve 301 and/or overmolds 403, 403′ may be elastic and/or flexible materials, for example, comprising a biocompatible polymer, so that sleeve 301 and/or overmolds 403, 403′ may stretch and/or compress as needed to permit movement of first and second arms 220, 222. A material of sleeve 301 and/or overmolds 403, 403′ may include silicones, urethanes, and/or nylons. Sleeve 301 and/or overmolds 403, 403′ may bias second arm 222 to the closed configuration. Sleeve 301 and/or overmolds 403, 403′ may stretch as instrument 232 and/or tube 290 is moved distally within passageway 275 (see FIGS. 3D and 4) and compress once the force provided by instrument 232 and/or tube 290 is removed.

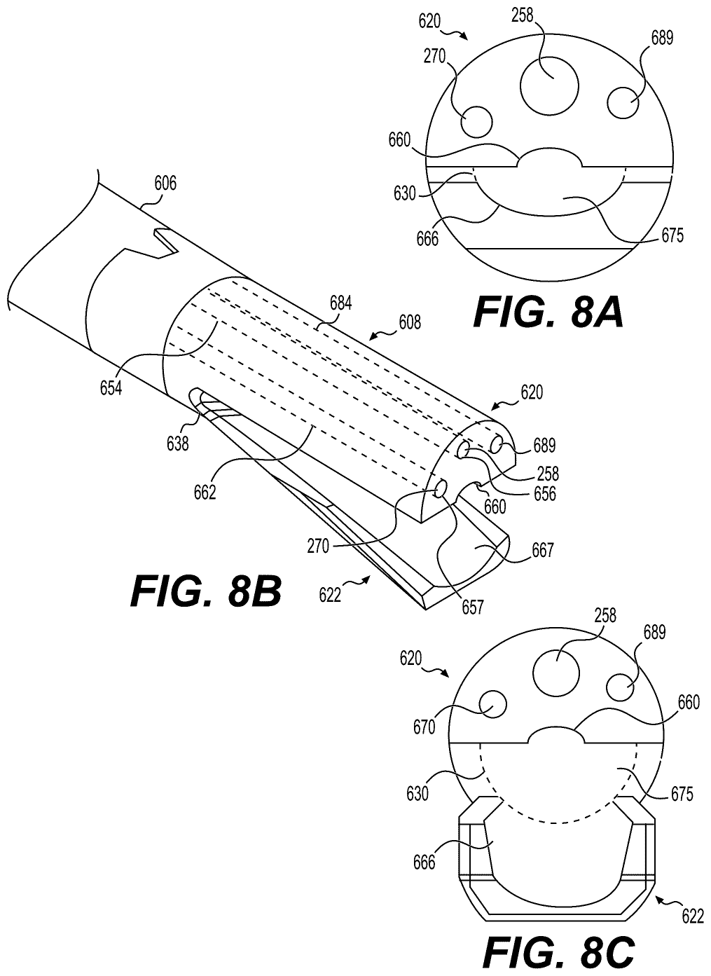

FIGS. 8A-8C illustrate additional features of the medical devices herein, e.g., shaft 606 and distal tip 608 that may be used in medical device 102 and that may include any of the features of shaft 206 and/or distal tip 208 discussed above. In this example, distal tip 608 includes at least one open lumen in a distalmost face of the medical device, e.g., useful for irrigation and/or suction.

Distal tip 608 is coupled to shaft 606 and includes a first arm 620 and a second arm 622 moveable relative to each other. For example, second arm 622 may be moveable relative to first arm 620 while first arm 620 is stationary relative to shaft 606. First arm 620 as shown includes three lumens 654, 662, 684 in communication with respective lumens in shaft 606. Each lumen 654, 662, 684 may extend from a proximal end of distal tip 608 to a distal end of first arm 620 (for an open lumen) or proximate the distal end of first arm 620 (for a closed lumen). For example, the lumens terminate at respective positions 656, 657, 689 of first arm 620, wherein each position may be an opening or the location of an electronic component, such as an imaging device, light source, or sensor, etc. The lumens may provide capability to convey electronic components to electronic components of distal tip 608 (e.g., the lumen terminating at an imaging device, light source, or sensor, etc. at the distalmost face of distal tip 608), or may be open to provide for irrigation and/or suction. For example, lumen 654 may have a closed end 656 that includes an imaging device 258 (e.g., camera, imager, etc.) to capture images of a treatment site. Further, for example, lumen 662 may have a closed end 657 that includes a light source 270 and lumen 684 may terminate in opening 689 to allow for delivery of fluid to a treatment site. For example, fluid may be introduced via a port of a handle (e.g., port 114 of handle 104 in FIG. 1) through a lumen in shaft 606 in communication with lumen 684 to exit through opening 689.

Similar to second arm 220, second arm 620 may include a channel 666. The edges of channel 666 may be further spaced apart than the edges of channel 660 of first arm 620. In other words, channel 666 may be wider than channel 660. In some examples, the width of channels 660, 666 may be the same. Channels 660, 666 form a passageway 675 similar to passageway 275. Similar to the mechanisms described above, distal movement of an instrument and/or a retractable tube through an opening 630, and through passageway 675 may move second arm 622 away from axis A to an open configuration and proximal movement through passageway 675 may move second arm 622 towards axis A to a closed configuration. In this example, second arm 220 is shown without lumens or electronic components, although it is contemplated that second arm 220 may include one or more lumens (closed or open-ended) and/or one or more electronic components (e.g., imaging device, light source, sensor, etc.).

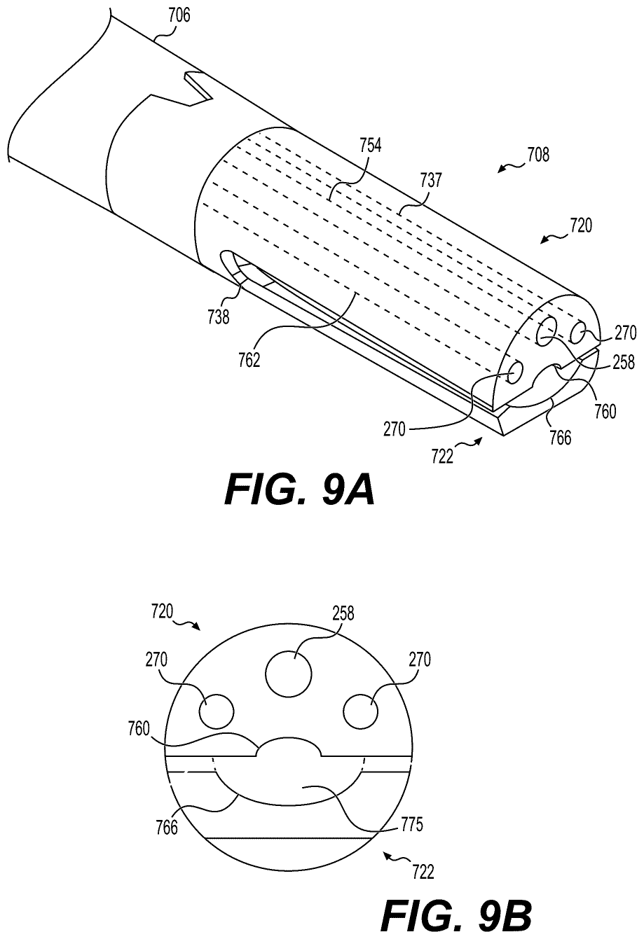

FIGS. 9A and 9B illustrate another exemplary distal end portion of a medical device according to aspects of the present disclosure, the distal end portion including a shaft 706 and a distal tip 708, which may be used in combination with any of the features of medical device 102 and/or combination with features of shafts 206, 606 and/or distal tips 208, 608. Distal tip 708 may include a first arm 720, a second arm 722, a hinge 738 allowing movement of second arm 722 relative to first arm 720, the first and second arms 720, 722 including channels 760, 766 and defining a passageway 775 therebetween. First arm 720 as shown includes lumens 754, 762, 737 in communication with respective lumens in shaft 706. Each lumen may be closed (e.g., terminating in a light source, imaging device, sensor, or other electronic component) or may be open (e.g., allowing for delivery of fluid and/or application of suction). In some examples, lumens 737, 762 are closed at the distal end of distal tip 708, e.g., each terminating in a light source 270, and lumen 754 is likewise closed at the distal end of distal tip 708, e.g., terminating in imaging device 258.

Similar to the mechanisms described above, distal movement of an instrument and/or a retractable tube through distal tip 708 through passageway 775 may apply a force to second arm 722 causing second arm 722 to move away from axis A and away from first arm 720 from a first, closed configuration to a second, open configuration. Similarly, proximal movement of the instrument and/or tube through passageway 775 may remove the force to cause movement second arm 722 towards axis A and towards first arm 720 to the first, closed configuration.

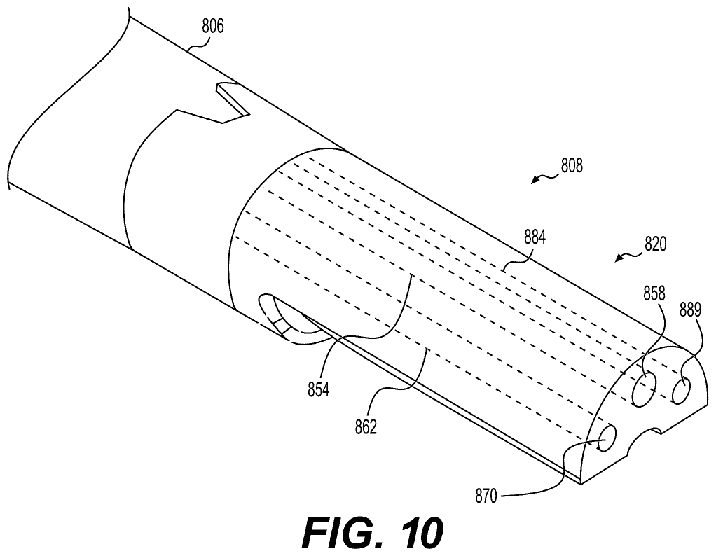

In some aspects of the present disclosure, the distal tip of the medical device includes only one arm. FIG. 10 illustrates a distal end portion of such a device, including shaft 806 and distal tip 808, where distal tip 808 includes arm 820. Distal tip 808 is similar to distal tip 708 of FIGS. 9A and 9B but lacking a second arm. For example, the portion of distal tip 808 opposite first arm 820 may include an open space wherein the working channel (e.g., working channel 228 of FIGS. 2A and 3A) is unobstructed. In this instance, distal tip 808 does not transition between different configurations, arm 820 having a fixed position relative to shaft 806. As shown, arm 820 includes lumens 854, 862, 884 in communication with respective lumens in shaft 806, lumen 854 being closed at the distal end and terminating in an imaging device 858, lumen 862 being closed at the distal end and terminating in light source 870, and lumen 884 being open at the distal end and terminating in opening 889 that may be used for delivery of fluid and/or suction.

It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and embodiments be considered as exemplary only.

1. A medical device comprising:
a shaft defining at least one working channel extending from a proximal end of the shaft to a distal end of the shaft along a longitudinal axis, the working channel having a cross-sectional dimension that allows passage of an instrument therethrough; and
a distal tip coupled to the shaft and comprising a first arm and a second arm, the first arm being moveable relative to the second arm between a first configuration that obstructs a portion of the working channel and a second configuration that provides unobstructed access to the working channel to allow the instrument to extend distal to the medical device.

2. The medical device of claim 1, wherein a cross-sectional dimension of the working channel at the distal end of the shaft is at least 50% of a cross-sectional dimension of the distal end of the shaft.

3. The medical device of claim 1, wherein the first arm is moveable relative to the second arm via a hinge.

4. The medical device of claim 3, wherein the hinge includes a leaf spring or a torsion spring.

5. The medical device of claim 3, wherein the hinge is a living hinge, the first arm being integral with the second arm and moveable via the living hinge.

6. The medical device of claim 1, wherein the distal tip is biased to the first configuration.

7. The medical device of claim 1, wherein the distal tip includes at least one of an imaging device or a light source and the shaft further comprises at least one lumen containing an electronic connection to the at least one of the imaging device or light source.

8. The medical device of claim 7, wherein the at least one of the imaging device or light source is on a distal-most surface of the distal tip.

9. The medical device of claim 1, wherein one of the first arm or the second arm includes an imaging device and the other of the first arm or the second arm includes a light source.

10. The medical device of claim 1, wherein the shaft further comprises a lumen for irrigation or suction, the lumen extending through the distal tip to a distal opening in the first arm or the second arm.

11. The medical device of claim 1, wherein the cross-sectional dimension of the working channel is at least 0.1 cm.

12. The medical device of claim 1, wherein a cross-sectional dimension of the shaft is greater than 0.1 cm and less than 0.3 cm.

13. The medical device of claim 1, wherein the working channel is defined by a tube moveable relative to the shaft and the distal tip.

14. The medical device of claim 1, further comprising a flexible or elastic sleeve around at least a portion of the distal tip.

15. The medical device of claim 1, wherein the distal tip is detachable from the shaft via complementary mating features of the distal tip and the shaft.

16. A medical device comprising:
a shaft defining at least one working channel extending along a longitudinal axis from a proximal end of the shaft to a distal end of the shaft; and
a distal tip coupled to the shaft and comprising a first arm and a second arm, the first arm being moveable relative to the second arm via a hinge between a first configuration that obstructs a portion of the working channel and a second configuration that provides unobstructed access to the working channel to allow an instrument to extend distal to the medical device, the distal tip being biased to the first configuration.

17. The medical device of claim 16, wherein the working channel extends through the distal tip between the first arm and the second arm.

18. The medical device of claim 16, wherein the distal tip includes an imaging device and a light source each disposed on a distal-most surface of the distal tip.

19. A medical device comprising:
a shaft defining at least one working channel extending from a proximal end of the shaft to a distal end of the shaft; and
a distal tip coupled to the shaft and comprising a first arm and a second arm, wherein each of the first arm and the second includes one of a light source or an imaging device at a distal end of the respective first or second arm, and wherein the first arm is moveable relative to the second arm between a first configuration that obstructs a portion of the working channel and a second configuration that provides unobstructed access to the working channel.

20. The medical device of claim 19, further comprising a flexible or elastic sleeve around at least a portion of the distal tip.
